(19) United States
(12) Patent Application Publication
(10) Pub. No.: US 2020/0085609 A1
(43) Pub. Date: Mar. 19, 2020

(54) DEVICE FOR THE HYGIENIC DISPOSAL OF HUMAN EXCRETIONS

(71) Applicant: PRO AQUA DIAMANTELEKTRODEN PRODUKTION GMBH & CO KG, Niklasdorf (AT)

(72) Inventors: Michael SCHELCH, Oberaich (AT); Wolfgang STABER, Bruck an der Mur (AT); Robert HERMANN, Oberaich (AT); Wolfgang WESNER, Wien (AT)

(73) Assignee: PRO AQUA DIAMANTELEKTRODEN PRODUKTION GMBH & CO KG, Niklasdorf (AT)

(21) Appl. No.: 16/468,560

(22) PCT Filed: Dec. 11, 2017
(86) PCT No.: PCT/EP2017/082287

§ 371 Date: Jun. 11, 2019

(30) Foreign Application Priority Data
Dec. 14, 2016 (AT) A51137/2016

(51) Int. Cl. A61F 5/442; A41D 1/06; A61F 5/455

(52) U.S. Cl. CPC A61F 5/442; A41D 2500/50; A61F 5/455; A41D 1/06

(57) ABSTRACT

Apparatus for the hygienic disposal of human excretion products, having pants made of a gas- and liquid-tight and elastic material having a waistband and leg bands having inner side inserts made of open-cell foam, the pants having an inner side covered with open-cell foam and/or elastic nubs, being provided with at least one suction hose connectable to a suction device or being able to be connected to such and having a device for introducing liquid.

The invention relates to an apparatus for the hygienic disposal of human excretion products, which apparatus has pants made of a gas- and liquid-tight and elastic material having a waistband and leg bands, wherein the pants are provided with at least one suction hose connectable to a suction device or can be connected to such.

Individuals who, due to their stage of development, physical condition or other reasons, such as wearing a protective suit, are unable to use toilets or to relieve themselves in the open over a long period of time, have up to now had to rely on wearing diapers. Diapers represent a close-to-body storage system for excrements and possibly also menstrual blood for a defined, limited period of time. The sorption media used in diapers have indeed already reached a very high level of development, if a timely changing, however, is not possible or is not performed, under certain circumstances, over a relatively long period, the bacteria contained in the diaper or developing there remain in contact with the skin of the wearer. Especially for adults, the use of diapers is associated with very unpleasant sensations during wear. Also, the changing of the diapers, especially by nursing staff, is perceived as unpleasant.

An apparatus of the type mentioned above is known, for example, from US 2004/0143229 A1. This apparatus designed as pants is only suitable for collecting and draining liquid excretion products and for the need of improving wearing comfort.

The invention has the object of providing an apparatus worn on the body for the hygienic disposal of human excretion products such as excrement and menstrual blood, which apparatus can be worn over a long period of time, is not associated with the disadvantages of diapers and guarantees a high wearing comfort.

This object is achieved according to the invention in that the pants have an inner surface covered with open-cell foam and/or elastic nubs and a device for introducing liquid, and wherein the waistband and the leg bands have inner side inserts made of open-cell foam.

The nubs or the foam on the inner side of the pants create a defined and permeable space between the skin and the pants. This makes it possible to introduce air or other gas through the open-cell foam at the waistband and the leg bands in the interior of such that no liquid passes from the inside to the outside and that a leading-away of excrement and the suction of the air/gas can be performed via the suction hose. In this case, both water and a cleaning liquid can be introduced into the interior via the device for introducing liquid, in order to promote the evacuation of excretion products and to perform a cleaning and disinfection of the skin. The air or gas stream then ensures the drying of the skin.

The apparatus is particularly suitable for the use by people who are not able to use toilets and who otherwise rely on the wearing of diapers, especially over a longer period of time, with all the disadvantages.

In a preferred embodiment of the apparatus, the device for introducing liquids has annular nozzles positioned in or on the inserts made of open-cell foam. In each case, a circumferential annular nozzle is located in each insert, which annular nozzle ensures optimal introduction of liquids into the interior of the pants to support the discharge of excretion products and for disinfecting and cleaning the interior and the skin of the wearer.

In a further preferred embodiment, the apparatus also has a device for introducing air or a gas, wherein this device comprises further annular nozzles in the region of the waistband and the leg bands, which further annular nozzles are positioned outside the annular nozzles provided for introducing liquids. Such a device is primarily provided when a sufficient air supply via the ambient air is not guaranteed, which may be the case, for example, in bedridden persons or persons in which the apparatus according to the invention is part of a protective suit which is worn over a longer period of time.

The supply of liquid and of air or gas to the relevant annular nozzles can be carried out in a simple manner via at least one supply hose and optionally at least one distribution hose. Distribution hoses are particularly advantageous for the connection of the individual annular nozzles to each other and can be guided along the outer side or the inner side of the pants.

The inserts made of open-cell foam preferably have a width of 1.0 cm to 5.0 cm, their thickness may be 0.1 cm to 5.0 cm, in particular 0.5 cm to 1.0 cm. The open-cell foam provided for the inserts and possibly for the inner side is preferably made of a durable and skin-friendly material, in particular polypropylene or polyethylene.

In a preferred embodiment, the nubs provided on the inner side of the pants consist of the material of the pants and are preferably formed together in the production of pants. The nubs can have a height of 0.5 cm to 3.0 cm, in particular up to 1.0 cm.

The design of the nubs themselves can be done in different ways. Preferred is an embodiment in which the nubs are designed as elongated elements having a rounded tip, similar to cartridge cases, or tapered, hemispherical and the like.

Also preferred is an embodiment in which the pants, possibly together with the nubs, are made of an elastomer, in particular of a rubber material or of plastic, or of silicone.

In particular, in a proposed use of the apparatus in protective suits, it is advantageous when the apparatus has a separating device for separating the gases contained in the suctioned mixture, liquids and solids as well as a storage for receiving the separated liquid and/or the separated solids. When used in a protective suit, it is also advantageous when the apparatus according to the invention has a device for processing the separated gas for its reuse.

Further features, advantages and details of the invention are now described with reference to the schematic drawing illustrating an embodiment in more detail. Shown are

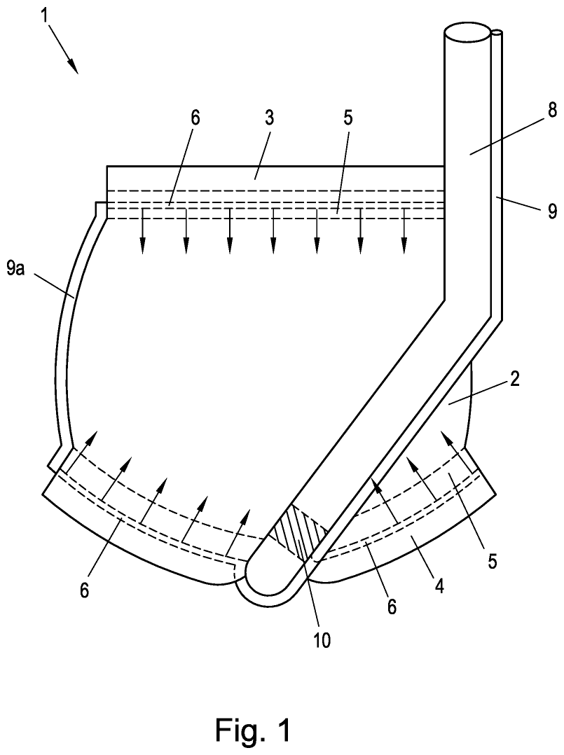

FIG. 1 an external view of pants belonging to an apparatus according to the invention and

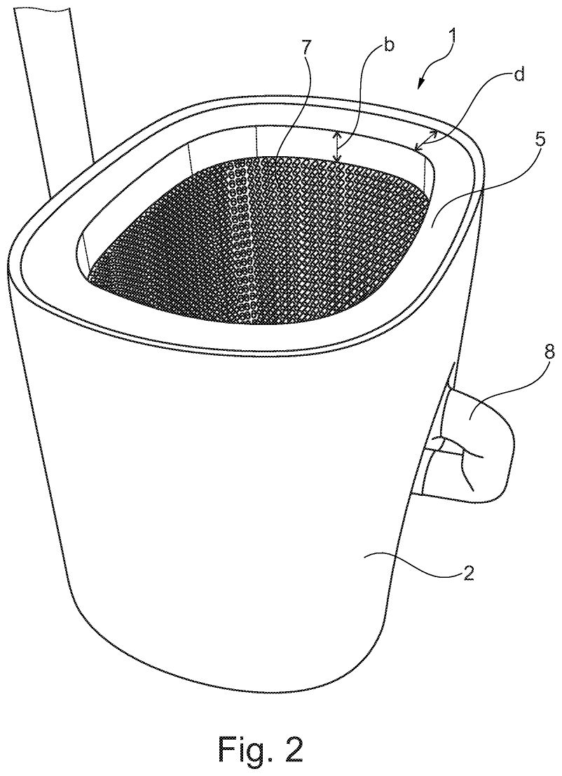

FIG. 2 an oblique view of the pants having a suction hose but without further hoses or supplies.

The apparatus according to the invention according to FIGS. 1 and 2 has pants 1 which are designed either leg-free (FIG. 1) or only provided with short legs. The pants 1 have an outer sheathe 2, which consists of a gas- and liquid-tight and elastic and skin-friendly material, preferably made of an elastomer, in particular of a rubber material or plastic, or of silicone. Rubber material is understood as meaning any suitable vulcanized rubber mixture, wherein it is possible in particular to use natural rubber and/or polyisoprene as rubber/rubbers. The pants 1 furthermore have a waistband 3 and two leg bands 4, each of which is formed by an insert 5 (FIG. 2) which encircles the inner side of the outer sheathe 2, made of an open-cell plastic foam, for example, made of polypropylene or polyethylene. The width b of the inserts 5 is preferably 1.0 cm to 5.0 cm, the thickness d of the inserts 5 preferably 0.1 cm to 3.0 cm, preferably up to 1.0 cm. Air or other gas can flow into the interior of the pants 1 through the inserts 5, so that no liquid can penetrate through the open-cell foam to the outside.

As shown particularly in FIG. 2, the inner side of the outer sheathe 2 is covered with a plurality of nubs 7, which preferably consist of the gas- and liquid-tight and elastic and skin-friendly material of the outer sheathe 2. The nubs 7 are in particular conical having a rounded tip and have, for example, a height of 0.5 cm to 3.0 cm, wherein all nubs 7 can be designed the same. In certain regions of the inner side of the outer sheathe 2, however, lower or higher nubs 7 may be provided than in the majority of the inner side. The nubs 7 can further have any cross-section provided with rounded tip and can be arranged such to immediately adjoin one another or be positioned at mutual distances of a few millimeters, wherein also in this regard in certain areas of the inner side of the outer sheathe 2, a greater or even lower density of nubs 7 than in the majority of the inner side can be provided. A volume-defined, permeable space is created between the skin of the wearer and the inner side of the outer sheathe 2 of the pants 1 through the nubs 7, wherein the arrangement of the nubs 7 is further such that as uniform as possible flow with air/gas is possible over the entire interior of the pants 1.

As an alternative to the nubs, the inner side can be covered with open-cell foam, analogous to the foam of the inserts 5 and in a thickness which corresponds to the thickness of the inserts 5. The foam layer can also be adapted to the body contours of the wearer.

An annular nozzle 6 each is positioned in the inserts 5 made of open-cell foam, the provided openings of said annual nozzles pointing in the direction of the flow-through interior of the pants 1, as indicated with arrows in FIG. 1. A supply hose 9 and at least one distribution hose 9a provide for feeding all of the annular nozzles 6 with liquid, as will be described. The pants 1 are further connected to a suction hose 8, which is preferably in spatial proximity to the anus and extends to the front side of the pants 1. A shredding device 10 for feces is location within the suction hose 8 in the vicinity of the suction point of the suction hose 8, so that said feces can be easily evacuated. In a simple embodiment, the shredding device 10, for example, consists of two crosswise arranged cutting blades. The suction hose 8 is connected to a suction device when the pants 1 are used, which suction device also provides for the leading-away of the air flowing through the inserts 5 into the interior of the pants 1. When using the pants 1 in regions with ambient air, such as in hospitals or nursing homes, the supplied air can come from the ambient air; in many cases, such as when used in a protective suit, a separate air/gas supply device is provided. The air/gas supply can also be carried out via further annular nozzles which extend in the region of the waistband and the leg bands in each case outside the annular nozzles 6. In this case, the outer sheathe 2 is guided at the edge over the inserts 5 in order to achieve a tight closure of the pants 1 to the skin of the wearer. The further annular nozzles can be introduced between the material of the outer sheathe extending over the outer sides of the inserts and the respective insert 5 and embedded in the foam material.

A timely leading-away of excretion products can be provided by means of the pants 1 according to the invention, in which a suction is performed via the suction hose 8, if necessary. Immediately before the excretion, the suction is put into operation to ensure a uniform flow of air through the interior. Simultaneously or immediately after the beginning of the suction, cleaning liquid is conducted via the annular nozzles 6 into the interior of the pants 1 and also suctioned. Preferably, water or a basic solution for disinfection and cleaning is suitable as a cleaning liquid. Particularly preferred is an alkaline solution having a pH of 11.0 to 12.0. Cleaning agents such as surfactants or disinfectants can be added to the cleaning fluid. The air flowing through the pants 1 or the gas flowing through the pants 1 must ensure that all liquids are transported safely and exclusively in the intended direction, in the direction of the suction hose 8.

The mixture of air, cleaning fluid and feces suctioned from the pants 1 is preferably first subjected to a separation of substances, wherein the liquid with the suspended or dissolved solids is separated from the air. A processing of the air or the gas can be carried out with one of the known cleaning methods. The fecal liquid is optionally disposed of after intermediate storage in a container via the sewer or alternatively processed analogously to the cleaning liquid.

The intended suction of the pants 1 can be carried out by an event-controlled regulation, for example, by triggering by the wearer, or be carried out in a permanent operation, for example, in the case of persons in high need of care. In nursing homes or other facilities where a high density of use of pants is expected, the suction can preferably be performed via a central suction system to which the individual pants are connected, for example, via hose coupling systems.

For example, discharge of excretion products can comprise the following sequential steps:
switching on the suction for the introduction of air/gas
adding clean water to the air stream via the annular nozzles 6 to allow a safe evacuation of the feces,
adding cleaning fluid to the air stream via the annular nozzles 6 to perform cleaning and disinfection of the skin,
adding clean water to the permanent air stream via the annular nozzles 6 to rinse off the cleaning agent,
final supplying of air without liquid to dry the skin.

These steps can be performed in a continuous sequence in a permanent operation of the pants 1.

If the apparatus is used by persons in protective suits, all units required for their operation can be accommodated in the protective suit itself.

### REFERENCE NUMBER LIST

1 . . . pants

2 . . . outer sheathe

3 . . . waist band

4 . . . leg band

5 . . . insert

6 . . . annular nozzle

7 . . . nubs

8 . . . suction hose

9 . . . supply hose

9a . . . distribution hose

10 . . . shredding device

1. An apparatus for the hygienic disposal of human excretion products, comprising:
pants made of a gas- and liquid-tight and elastic material having a waistband and leg bands, the pants being provided with at least one suction hose connectable to a suction device or can be connected to such, wherein:
the pants have an inner side covered with open-cell foam and/or elastic nubs and a device for introducing liquid, and
the waistband and the leg bands have inner side inserts made of open-cell foam.

2. The apparatus according to claim 1, wherein the device for introducing liquids has annular nozzles which are positioned in or on the inserts made of open-cell foam.

3. The apparatus according to claim 1, further comprising a device for introducing air or gas by means of further annular nozzles positioned in the region of the waistband and the leg bands, which further annular nozzles extend outside of the annular nozzles provided for introducing liquids.

4. The apparatus according to claim 1, wherein the device for introducing liquid and those for the introducing air or gas in each case have at least one supply hose and optionally at least one distribution hose.

5. The apparatus according to claim 1, wherein the inserts consisting of open-cell foam have a width of 1.0 cm to 5.0 cm.

6. The apparatus according to claim 5, wherein the open-cell foam has a thickness of 0.1 cm to 5.0 cm.

7. The apparatus according to claim 5, wherein the open-cell foam consists of polypropylene or polyethylene.

8. The apparatus according to claim 1, wherein the nubs are made of the material of the pants.

9. The apparatus according to claim 1, wherein the nubs have a height of 1.0 cm to 3.0 cm, in particular up to 1.0 cm.

10. The apparatus according to claim 1, wherein the nubs are designed conically having a rounded tip.

11. The apparatus according to claim 1, wherein the pants and the nubs are made of an elastomer, in particular of a rubber material or of plastic, or of silicone.

12. The apparatus according to claim 1, further comprising a separating device for separating the gases, liquids and solids contained in the suctioned mixture.

13. The apparatus according to claim 12, further comprising a storage for receiving the separated liquid and/or the separated solids.

14. The apparatus according to claim 12, further comprising an apparatus for processing the separated gas for its reuse.

15. The apparatus according to claim 12, further comprising a storage for receiving the separated liquid and/or the separated solids.
